(19) United States
(12) Patent Application Publication
(10) Pub. No.: US 2024/0148328 A1
(43) Pub. Date: May 9, 2024

(54) INTELLIGENT IMPLANTS

(71) Applicant: NuVasive, Inc., San Diego, CA (US)

(72) Inventors: Shane Fedon, Elfin Forest, CA (US); Mike Serra, San Diego, CA (US); Robert German, San Diego, CA (US); Matthew Jacobs, San Diego, CA (US); Michael Brotman, San Diego, CA (US); Greg Marik, San Diego, CA (US); Jeremiah Beers, San Diego, CA (US)

(21) Appl. No.: 18/501,583

(22) Filed: Nov. 3, 2023

Related U.S. Application Data
(60) Provisional application No. 63/382,400, filed on Nov. 4, 2022.

(51) Int. Cl. A61B 5/00; A61B 5/0538; A61B 5/07; A61B 5/11; A61F 2/44

(52) U.S. Cl. CPC A61B 5/686; A61B 5/0538; A61B 5/076; A61B 5/1116; A61B 5/4851; A61F 2/4455; A61B 2560/0443; A61B 2562/0219; A61B 2562/0252; A61F 2002/3067

(57) ABSTRACT

An intervertebral implant includes a body adapted to be implanted into an intervertebral space of a patient, the body defining a fusion aperture, and at least one cartridge with each cartridge disposed in the body and at least partially extending into the fusion aperture. Each cartridge includes an impedance sensor configured to measure electrical resistance, a radio frequency (RF) transmit antenna configured to transmit a RF signal, and circuitry for recording data from the impedance sensor and transmit the recorded data to an external clinician computing device through the RF transmit antenna. Such collection and use of data can be used to provide improved orthopedic outcomes.

### CROSS-REFERENCE TO RELATED APPLICATIONS

The present application claims priority to U.S. Provisional Patent Application No. 63/382,400, filed on Nov. 4, 2022, which is incorporated herein by reference. The present application is also related to, but does not claim priority to U.S. patent application Ser. No. 18/477,841, filed on Sep. 29, 2023, which is also incorporated herein by reference.

### BACKGROUND

The spinal column is a highly complex system of bones and connective tissues that provide support for the body and protect the delicate spinal cord and nerves. The spinal column includes a series of vertebral bodies stacked atop one another, each vertebral body including an inner or central portion of relatively weak cancellous bone and an outer portion of relatively strong cortical bone. Situated between each vertebral body is an intervertebral disc that cushions and dampens compressive forces exerted upon the spinal column A vertebral canal containing the spinal cord is located behind the vertebral bodies. The spine has a natural curvature (i.e., lordosis in the lumbar and cervical regions and kyphosis in the thoracic region) such that the endplates of the upper and lower vertebrae are inclined towards one another.

There are many types of spinal column disorders including scoliosis (abnormal lateral curvature of the spine), excess kyphosis (abnormal forward curvature of the spine), excess lordosis (abnormal backward curvature of the spine), spondylolisthesis (forward displacement of one vertebra over another), and other disorders caused by abnormalities, disease, or trauma (such as ruptured or slipped discs, degenerative disc disease, fractured vertebrae, and the like). Patients that suffer from such conditions often experience extreme and debilitating pain, as well as diminished nerve function. Posterior fixation for spinal fusions, decompression, deformity, and other reconstructions are performed to treat these patients. The aim of posterior fixation in lumbar, thoracic, and cervical procedures is to stabilize the spinal segments, correct multi-axis alignment, and aid in optimizing the long-term health of the spinal cord and nerves.

Spinal deformity is the result of structural change to the normal alignment of the spine and is usually due to at least one unstable motion segment. The definition and scope of spinal deformity, as well as treatment options, continues to evolve. Surgical objectives for spinal deformity correction include curvature correction, prevention of further deformity, improvement or preservation of neurological function, and the restoration of sagittal and coronal balance. Sagittal plane alignment and parameters in cases of adult spinal deformity (ASD) are becoming increasingly recognized as correlative to health-related quality of life score (HRQOL). In the literature, there are significant correlations between HRQOL scores and radiographic parameters such as Sagittal Vertical Axis (SVA), Pelvic Tilt (PT) and mismatch between pelvic incidence and lumbar lordosis.

The SRS-Schwab classification of ASD was developed to assist surgeons with a way to categorize ASD, and provide methods of radiographic analysis. This classification system helps provide a protocol for pre-operative treatment planning and post-op assessment. The current environment to utilize this classification system requires surgeons to examine pre-operative patient films and measure pelvic incidence, lumbar lordosis, pelvic tilt, and sagittal vertical axis either manually or through the use of pre-operative software. After the procedure, the surgeon examines the post-operative films and measures the same parameters and how they changed as a result of the surgery. A need exists for systems and methods for assessing these and other spinal parameters intraoperatively and assessing changes to these intraoperative spinal parameters as a surgical procedure progresses towards a pre-operative plan.

During spinal surgeries, screws, hooks, and rods are devices used to stabilize the spine. Such procedures often require the instrumentation of many bony elements. The devices, for example rods, can be extremely challenging to design and implant into the patient. Spinal rods are usually formed of stainless steel, titanium, cobalt chrome, or other similarly hard metal, and as such are difficult to bend without some sort of leverage-based bender. Moreover, a spinal rod needs to be oriented in six degrees of freedom to compensate for the anatomical structure of a patient's spine as well as the attachment points (screws, hooks, etc.) for securing the rod to the vertebrae. Additionally, the physiological problem being treated as well as the physician's preferences will determine the exact configuration necessary. Accordingly, the size, length, and particular bends of the spinal rod depends on the size, number, and position of each vertebrae to be constrained, the spatial relationship amongst vertebrae, as well as the screws and hooks used to hold the rods attached to the vertebrae.

The bending of a spinal rod can be accomplished by a number of methods. The most widely used method is a three-point bender called a French Bender. The French bender is a pliers-like device that is manually operated to place one or more bends in a rod. The French bender requires both handles to operate and provides leverage based on the length of the handle. The use of the French bender requires a high degree of physician skill because the determination of the location, angle, and rotation of bends is often subjective and can be difficult to correlate to a patient's anatomy. Other methods of bending a rod to fit a screw and/or hook construct include the use of an in-situ rod bender and a keyhole bender. However, all of these methods can be subjective, iterative, and are often referred to as an “art.” As such, rod bending and reduction activities can be a time consuming and potentially frustrating step in the finalization of a complex and/or long spinal construct. Increased time in the operating room to achieve optimum bending can be costly to the patient and increase the chance of the morbidity. When rod bending is performed poorly, the rod can preload the construct and increase the chance of failure of the fixation system. The bending and re-bending involved can also promote metal fatigue and the creation of stress risers in the rod.

Efforts directed to computer-aided design or shaping of spinal rods have been largely unsuccessful due to the lack of bending devices as well as lack of understanding of all of the issues involved in bending surgical devices. Recently, in U.S. Pat. No. 7,957,831 to Isaacs, there is described a rod bending system which includes a spatial measurement sub-system with a digitizer to obtain the three-dimensional location of surgical implants (screws, hooks, etc.), software to convert the implant locations to a series of bend instructions, and a mechanical rod bender used to execute the bend instructions such that the rod will be bent precisely to custom fit within each of the screws. This is advantageous because it provides quantifiable rod bending steps that are customized to each patient's anatomy enabling surgeons to create custom-fit rods on the first pass, thereby increasing the speed and efficiency of rod bending, particularly in complex cases. This, in turn, reduces the morbidity and cost associated with such procedures. However, a need still exists for improved rod bending systems that allow for curvature and deformity correction in fixation procedures, provide the user with more rod bending options, and accommodate more of the user's clinical preferences.

### SUMMARY

An intervertebral implant includes a body adapted to be implanted into an intervertebral space of a patient, the body defining a fusion aperture, and at least one cartridge with each cartridge disposed in the body and at least partially extending into the fusion aperture. Each cartridge includes an impedance sensor configured to measure electrical resistance, a radio frequency (RF) transmit antenna configured to transmit a RF signal, and circuitry for recording data from the impedance sensor and transmit the recorded data to an external clinician computing device through the RF transmit antenna such that patient status after the surgical procedure can be monitored without any invasive procedure and corrected if necessary.

### BRIEF DESCRIPTION OF THE DRAWINGS

FIG. 1 illustrates an example smart implant system according to one aspect of the present invention.

FIG. 2 illustrates an example implant configured to couple a cartridge to another implant (e.g., a spinal rod).

FIG. 3 illustrates the implant of FIG. 2 with a cartridge received within the cartridge receiver and a rod received within the rod receiver.

FIG. 4 illustrates an example implant configured to couple a cartridge to another implant (e.g., a spinal rod).

FIG. 5 illustrates the implant of FIG. 4 with a cartridge received within the cartridge receiver and a rod received within the rod receiver.

FIG. 6 illustrates an example implant in the form of a spinal cross-connector being a cartridge or a cartridge being disposed within a spinal cross connector.

FIG. 7 illustrates the example implant of FIG. 6 in an exploded view.

FIG. 8 illustrates a longitudinal cartridge that is cantilevered into a fusion aperture.

FIG. 9 illustrates a longitudinal cartridge that extends entirely through the fusion aperture.

FIG. 10 illustrates a longitudinal cartridge that extends entirely through the fusion aperture and out another side of the implant.

FIG. 11 illustrates a transverse cartridge that is cantilevered into a fusion aperture.

FIG. 12 illustrates a transverse cartridge that extends entirely through the fusion aperture.

FIG. 13 illustrates a transverse cartridge that extends entirely through the fusion aperture and out another side of the implant.

FIG. 14 illustrates an oblique cartridge that is cantilevered into a fusion aperture.

FIG. 15 illustrates an oblique cartridge that extends entirely through the fusion aperture.

FIG. 16 illustrates an oblique cartridge that extends entirely through the fusion aperture and out another side of the implant.

FIG. 17 illustrates the cartridge being non-parallel to the length of the implant, such as the relative position with respect to the height of the implant varies along the length of the cartridge.

FIG. 18 illustrates the cartridge being non-parallel to the length of the implant, such as the relative position with respect to the width of the implant varies along the length of the cartridge.

FIG. 19 illustrates the cartridge being non-parallel to the length of the implant, such as the relative position with respect to the width and height of the implant varies along the length of the cartridge.

FIG. 20 illustrates the position of the cartridge being located near a portion of the implant having a greater height.

FIG. 21 illustrates an implant having a cartridge floating between a first endplate and a second endplate separated by struts.

FIG. 22 illustrates a top-down view of an arcuate cartridge disposed within an implant.

FIG. 23 illustrates a top-down view of an elongate cartridge with an arcuate portion disposed within an implant.

FIG. 24 illustrates a top-down view of an implant having an elongate cartridge with first and second lobes separated by a connector.

FIG. 25 illustrates an example cartridge configured for insertion into a screw hole of an implant.

FIG. 26 illustrates an example anterior lumbar interbody fusion implant with bone screws and two cartridges in the form illustrated in FIG. 25 inserted.

FIG. 27 illustrates an example implant with a plate.

FIG. 28 illustrates an example implant with a cartridge coupled thereto in the form of a plate (e g, similar to plate with or without fixation apertures).

FIG. 29 illustrates a partial cutaway view of a cartridge.

FIG. 30 illustrates an example implant in a lateral view having anterior and posterior cutouts in anterior and posterior faces of the implant configured to receive one or more cartridges.

FIG. 31 illustrates a spinal fixation system benefitting from aspects disclosed herein.

FIG. 32 illustrates an example lateral spinal fusion implant.

FIG. 33 illustrates a lateral partial cross-sectional view of a prosthetic disc that can benefit from aspects disclosed herein.

FIG. 34a shows a single-level bone plate.

FIG. 34b shows a two-level bone plate.

### DETAILED DESCRIPTION

Smart technology can be used to improve orthopedic surgery, including spine surgery. Smart technology can include the collection and use of data to inform preoperative, intraoperative, and postoperative decisions. Sensors can be used to obtain data including accelerometers, gyroscopes, vibration sensors, piezoelectric devices configured as sensors, stress sensors, stain sensors, temperature sensors, position sensors, fiber bragg grating sensors, chemical sensors, optical sensors, biofilm sensors, sensors for detecting biofilm which include an RF transmitter and an RF receiver, other sensors, and combinations thereof.

### Intelligent Implant System

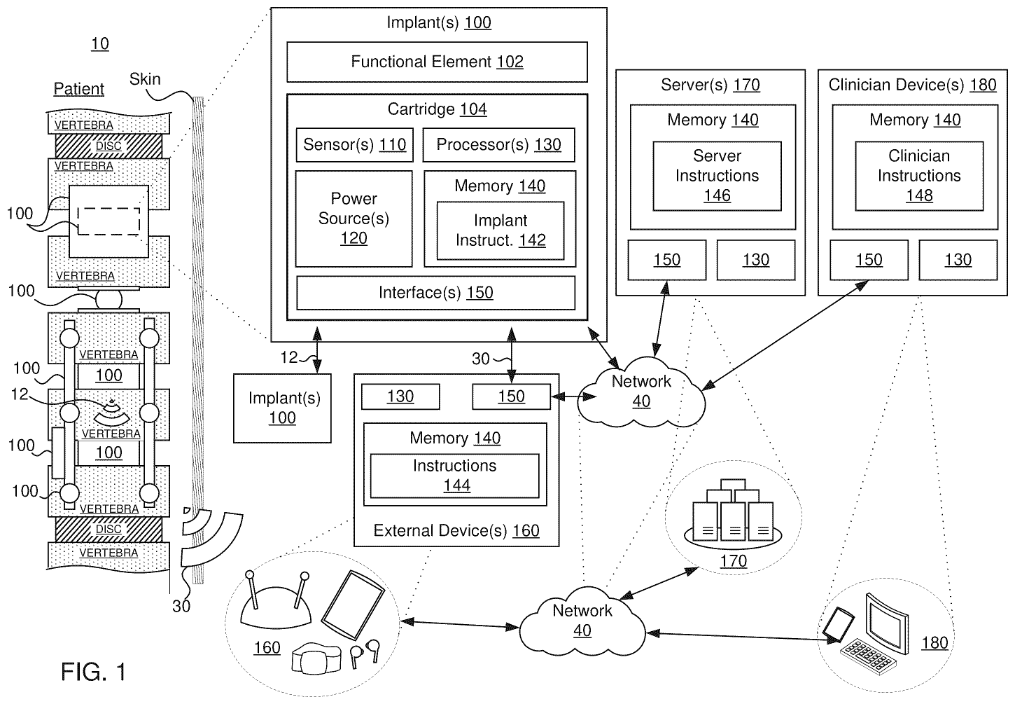

FIG. 1 illustrates an example smart implant system 10. As illustrated, the system 10 includes portions implanted in a patient and external to the patient. The portions implanted in the patient include a plurality of different implants 100. The portions external to the patient include one or more external devices 160, one or more servers 170, and one or more clinician devices 180 connected via a network 40. The network 40 is a group of communicatively coupled computing environments and associated hardware, such as a local area network, the Internet, other networks, or combinations thereof.

As illustrated, the implants 100 are primarily in the form of spinal implants, but aspects described herein can be relevant to other medical implants. Other example components include intramedullary devices, implanted stimulators, other devices, or combinations thereof. The implants 100 include: spinal fusion implants, intervertebral implants, total disc replacement implants, spinal plates, pedicle screws, spinal rods, structural rods, non-structural rods, spinous process implants, interlaminary spacers, standalone implants, rib-hook implants, piggyback implants, other implants, or combinations thereof. In an example, the implant 100 is a cannulated screw or spinal rod. There can be an electrically non-conductive polymer (e.g., HDPE) that is used as a plug to plug one or more cannulations or holes in an implant. In an example, the implant 100 is a spinal tethering device and one or more sensors 110 can sense tension on a tether.

The functional diagram of an example implant 100 illustrates that an implant can include one or more functional elements 102, one or more sensors 110, one or more power sources 120, one or more processors 130, memory 140 having implant instructions 142, one or more interfaces 150, other components, and combinations thereof. As illustrated, one or more of the components can be collected as part of a cartridge 104. Other example components include stimulators, drug delivery components, active components, or actuatable components. For ease of illustration, one implant 100 is shown as having all of those components, but in actuality the implants 100 need not have all of those components and one or more components can be allocated among different components. In some examples, an implant 100 can have a primary therapeutic function and a secondary “smart” function, a primary “smart” function with or without a primary or secondary therapeutic function.

The cartridge 104 can be a package, housing, device, or other container in which one or more components can be collected. The cartridge 104 can include the sensors 110, processor(s), power source(s) 120, memory 140, and interface(s) 150. The cartridge 104 can be a hermetically sealed component.

The cartridge 104 can be configured as an independent component from the implant 100. The cartridge 104 can then be inserted into or otherwise coupled with the implant 100. This can occur during a manufacturing process or in an operating room during surgery. The coupling can take any of a variety of forms, such as a threaded connection, a snap fit connection, a tortuous path, dovetail connection, other connections, or combinations thereof. In other examples, the cartridge 104 can be substantially integral with the implant 100.

The one or more functional elements 102 can be one or more components that provide therapeutic function. Examples of functional elements 102 include one or more structures that facilitate statically expanding an intervertebral disc space, dynamically expanding an intervertebral disc space, separate vertebral endplates, support vertebral implants, modify lordosis, modify kyphosis, replace a vertebral body, maintain a distance between anatomic features, securing the implant or another implant to a body, permit articulation, resist articulation, encourage bone growth, other functions, or combinations thereof. Example structural features that can be used to provide such features include but are not limited to: body material, struts, threads, fasteners, actuators, expanders, porous components, other structures, or combinations thereof.

The one or more sensors 110 are components that produce output based on measurements. Example sensors 110 include accelerometers, gyroscopes, vibration sensors, piezoelectric devices configured as sensors, stress sensors, stain sensors, temperature sensors, position sensors, fiber bragg grating sensors, chemical sensors, optical sensors, electrical sensors (e.g., one or more electrodes), electromagnetic sensors (e.g., antenna(s)), Hall effect sensors, other sensors, and combinations thereof. In an example, the sensors 110 can include a nine-axis inertial measurement unit. In an example, the sensors 110 can include a one-, two-, or three-axis accelerometer. In an example, the sensors 110 can include a one-, two-, or three-axis gyroscope. In an example, the sensors 110 can include a one-, two-, or three-axis magnetometer. The sensors 110 can produce output based on sound waves, ultrasonic waves, light, radiation, or electromagnetic field changes. The output of the sensors 110 can take any of a variety of useful forms, such as a variable voltage, a signal that encodes data, other forms, or combinations thereof. In some examples, the sensors 110 include some data processing components configured to modify “raw” data prior to output. Such components can include signal processing circuitry, amplifiers, filters, microcontrollers, processors, other components, or combinations thereof. In other examples, the sensors 110 can provide “raw” output that is processed by other components. In an example, the sensors 110 are or include one or more layers of conductive material or conductive traces. A capacitive or resistive circuit to measure the electrical impedance to determine that something has moved.

In an example implementation, the sensors 110 include a Hall Effect sensor. The Hall Effect sensor can be used as a trigger to determine if something has moved. For instance, a Hall Effect sensor can be used to detect a screw backing out or particular motion of a movable component. There can be a ferrite material placed in an implant or component being monitored for movement to improve detection by the Hall Effect sensor.

In an example, components of the implant 100 are turned into or include sensors 110, such as force sensors. For instance, the endplates of the implant 100 can be constructed from or include PEEK and silver (or other conductive components) is etched or otherwise added to create conductive strips across both endplates and use a capacitor (e.g., on a printed circuit board assembly) to measure pressure or capacitive changes that's a force sensor with multiple zones on bottom or top plate to measure loading.

The one or more power sources 120 are components are components configured to usefully power other components. The one or more power sources 120 can take any of a variety of forms, such as rechargeable batteries, non-rechargeable batteries, capacitors, other components or combinations thereof. The one or more power sources 120 can also include one or more components configured to receive or generate power. For example, the power sources 120 can include one or more components configured to receive wirelessly transmitted external power, such as through an inductive connection or through ultrasound transmission. The component can generate power, such as by harvesting energy from the recipient of the implant 100, such as via a piezoelectric or metamaterial component that generates power from movement of the recipient.

The one or more processors 130 are one or more physical or virtual components configured to obtain and execute instructions. In many examples, the one or more processors 130 are central processing units, but can take other forms such as microcontrollers, microprocessors, field programmable gate arrays, graphics processing units, tensor processing units, other processors, or combinations thereof.

The memory 140 is one or more physical or virtual components configured to store information, such as data or instructions. In some examples, the memory 140 includes a computing environment's main memory (e.g., random access memory) or long-term storage memory (e.g., a solid state drive). The memory can be transitory or non-transitory computer-readable or processor-readable storage media. The memory 140 can include read only or read-write memory.

The implant instructions 142 are one or more instructions that, when executed, cause the one or more processors 130 of the implant 100 to perform one or more operations. The operations can take any of a variety of forms, such as the receipt of data (e.g., from the one or more sensors 110), processing of data (e.g., in the memory 140), and communication of data (e.g., to another implant 100 or another device via the one or more interfaces 150). As illustrated, inter-implant communication 12 can be performed. Transcutaneous communication 30 can also be used.

The one or more interfaces 150 is a set of one or more components by which the implant 100 or components thereof can provide output or receive input. For example, the interface 150 can include one or more user input components, such as one or more sensors, buttons, pointers, keyboards, mice, gesture controls, touch controls (e.g., touch-sensitive strips or touch screens), eye trackers, voice recognition controls (e.g., microphones coupled to appropriate natural language processing components), other user input components, or combinations thereof. In some examples, the one or more interfaces 150 can be directly actuatable transcutaneously (e.g., pressing a button through the skin of the patient) or an external device having a connection to the implant 100 (e.g., through a wireless radiofrequency connection that connects a keyboard or other device to the interfaces 150). The interface 1030 can include one or more user output components, such as one or more lights, displays, speakers, haptic feedback components, other user output components, or combinations thereof. The interface 1030 can further include one or more components configured to provide output to or receive input from other devices (e.g., implanted or external devices), such as one or more ports (e.g., USB ports, THUNDERBOLT ports, serial ports, parallel ports, Ethernet ports) or wireless communication components (e.g., components configured to communicate according to one or more radiofrequency protocols, such as WI-FI, BLUETOOTH, ZIGBEE, or other protocols). In an example, the interface 150 includes one or more components for use in ultrasound communication to receive or transmit ultrasound signals. Example ultrasound communication techniques are described in US20200253588 (application Ser. No. 16/785,240, filed 2020 Jan. 10) which is hereby incorporated herein by reference in its entirety for any and all purposes.

In an example, the interface 150 includes an antenna for communication. In an example, an antenna of the interface 150 can be wrapped around the implant 100, the cartridge 104, or a portion thereof. In an example, this can be performed during the manufacture of the implant 100 or during assembly of the cartridge 104 into the implant 100. In an example, there is a multi-step 3D printing process where we injection mold an antenna into the outer structure or endplates. In an example, there can be a wire wrapped around a spinal rod to reduce interference. There can be an exterior surface of an implant or a component that would become an antenna with less interference. There can be a small piece of wire (e.g., gold wire) acting as an antenna. One or more of the antenna wires can be epoxy bonded to a component or implant (e.g., one or more spinal rods or a crosslink connector). There can be a plastic (e.g., PEEK) cap on the end of a rod and then from that it can be encapsulated as antenna as an injection mold piece.

External devices 160 are one or more devices local to the patient but external to the recipient's body. These can include a wide variety of devices, such as personal computing devices, hub devices, operating room devices, other devices, or combinations thereof. Example personal computing devices include cell phones, smart phones, smart watch, smart earbuds, tablet, personal computer, laptop, headphones, personal health device (e.g., heartrate monitor, blood pressure cuff, therapy device), smart ring, sleep tracker, sleep apnea device, other devices, or combinations thereof.

The servers 170 are one or more computing devices remote from the implants 100, external devices 160, and clinician devices 180. Nonetheless, the servers 170 can be indirectly communicatively connected to the implants 100, external devices 160, and clinician devices 180 via the network 40. As illustrated, the servers 170 can be computing devices that include one or more processors 130, memory 140, and interfaces 150. These components can be similar to those described elsewhere herein but customized for their specific use and environment. The servers 170 can include server instructions 146 that, when executed by the one or more processors 130 of the servers 170 cause the processors 130 to perform one or more operations.

The clinician devices 180 are one or more computing devices used by medical professionals that provide, maintain, or support the implants 100 or functions thereof. The clinician devices 180 can be directly or indirectly communicatively connected to the implants 100, external devices 160, and servers 170 via the network 40. As illustrated, the clinician devices 180 can be computing devices that include one or more processors 130, memory 140, and interfaces 150. These components can be similar to those described elsewhere herein but customized for their specific use and environment. The memory 140 of the clinician devices 180 can include clinician instructions 148 that, when executed by the one or more processors 130 of the clinician devices 170 cause the processors 130 to perform one or more operations. For example, the operations can include operations associated with one or more software applications that monitor or support the patient via data sensed by the one or more implants 100.

### Implant-Cartridge Connection

FIGS. 2-21 illustrate various implementations of connections between implants 100 and cartridges 100.

FIG. 2 illustrates an example implant 200 configured to couple a cartridge 104 to another implant 100 (e.g., a spinal rod). The implant 200 includes a rod receiver 210 and a cartridge receiver 212. The rod receiver 210 having an opening 210a that allows a spinal rod to pass through and a set screw opening 210b for fixing the position of the implant 200 relative to the another implant 100 (e.g., a spinal rod). The cartridge receiver 212 is configured to receive and retain the cartridge 104.

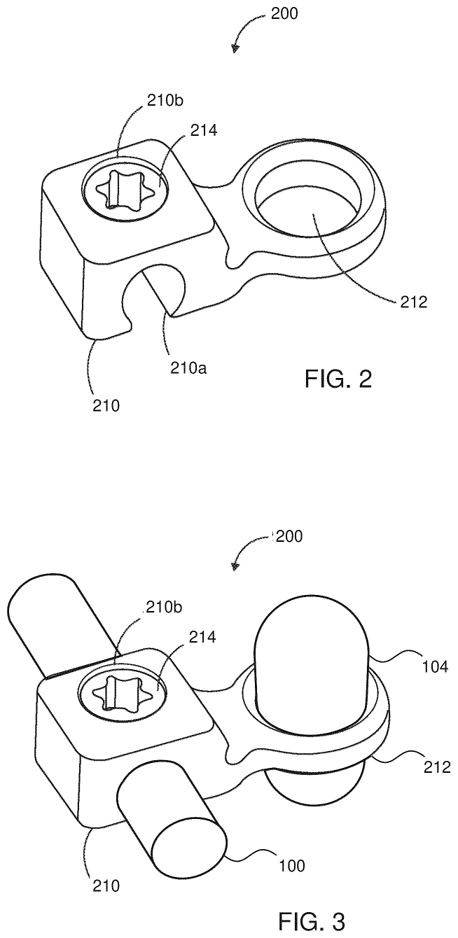

FIG. 3 illustrates the implant 100 of FIG. 2 with a cartridge 104 received within the cartridge receiver 212 and a rod 100 received within the rod receiver 210. As illustrated, the implant 200 holds the rod 100 and cartridge 104 such that the rod 100 and cartridge 104 are held perpendicular relative to each other. Generally, the rod 100 would extend cranially-caudally within a patient. Generally, an elongate cartridge 104 can extend generally superficial-deep, anterior-posterior, or laterally with respect to the patient's anatomy. This implementation is illustrated as having a fixed connection between the rod receiver 210 and the cartridge receiver 212, but the connection need not be fixed. For example, the components can be configured to be angled relative to each other (e.g., to facilitate placement relative to the patient's anatomy) and then have the angle locked.

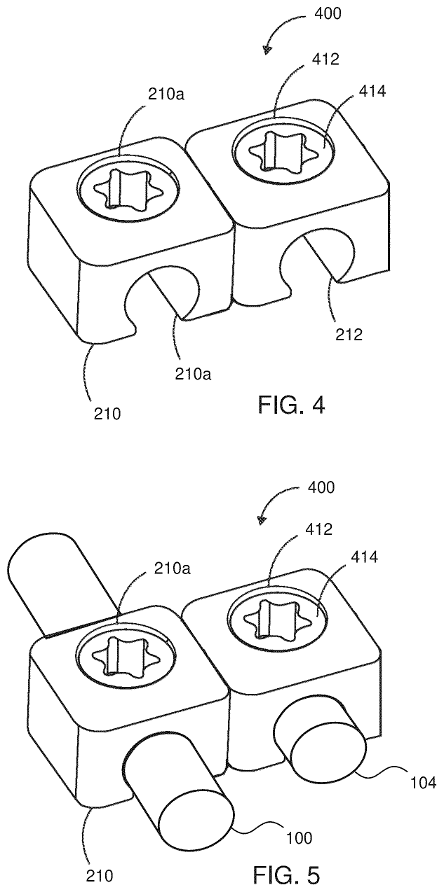

FIG. 4 illustrates an example implant 400 configured to couple a cartridge 104 to another implant 100 (e.g., a spinal rod). The implant 200 includes a rod receiver 210 and a cartridge receiver 212. The rod receiver 210 having an opening 210a that allows a spinal rod to pass through and a set screw opening 210b for fixing the position of the implant 200 relative to the another implant 100 (e.g., a spinal rod). The cartridge receiver 212 is configured to receive and retain the cartridge 104. In this implementation, the implant 400 is configured to retain the cartridge 104 substantially parallel to the spinal rod 100. The implant 400 further includes a set screw opening 412 and set screw 414 configured to interact with and retain the cartridge within the cartridge receiver 212.

FIG. 5 illustrates the implant 400 of FIG. 4 with a cartridge 104 received within the cartridge receiver 212 and a rod 100 received within the rod receiver 210. As illustrated, the implant 200 holds the rod 100 and cartridge 104 such that the rod 100 and cartridge 104 are held substantially parallel. This implementation is illustrated as having a fixed connection between the rod receiver 210 and the cartridge receiver 212, but the connection need not be fixed. For example, the components can be configured to be angled relative to each other (e.g., to facilitate placement relative to the patient's anatomy) and then have the angle locked.

In an example, the implants 200, 400 of FIGS. 2-5 are used in a method for implanting a cartridge 104. In an example, during a spinal fusion procedure, one or more pedicle screws are placed and one or more structural spinal rods are placed. For example, there can be a spinal fusion procedure in which pedicle screws are placed in vertebrae above and below the location that a spinal fusion implant is placed. The pedicle screws are then joined by spinal rods. The implants 200, 400 can be used to couple a cartridge 104 to the spinal rods. For example, after implantation of the rods, the implants 200, 400 can be coupled to the rods. In an example, the implants 200, 400 are coupled to the rods before, during, or after implantation of the spinal rods. The cartridge 104 can be coupled to the implants 200, 400 before, during, or after coupling the implants 200, 400 to the spinal rods. When coupling the cartridge 104 can be substantially perpendicular, parallel, or orthogonal to the spinal rod. In an example, the cartridge 104 is cantilevered. In another example, the cartridge 104 is supported at a plurality of locations. For instance, there can be two implants 400 each supporting a single cartridge 104. In an example, pedicle screws support the cartridge 104. For example, a structural spinal rod construct can include a first spinal rod coupled to two or more pedicle screws and a cartridge coupled to two or more other pedicle screws.

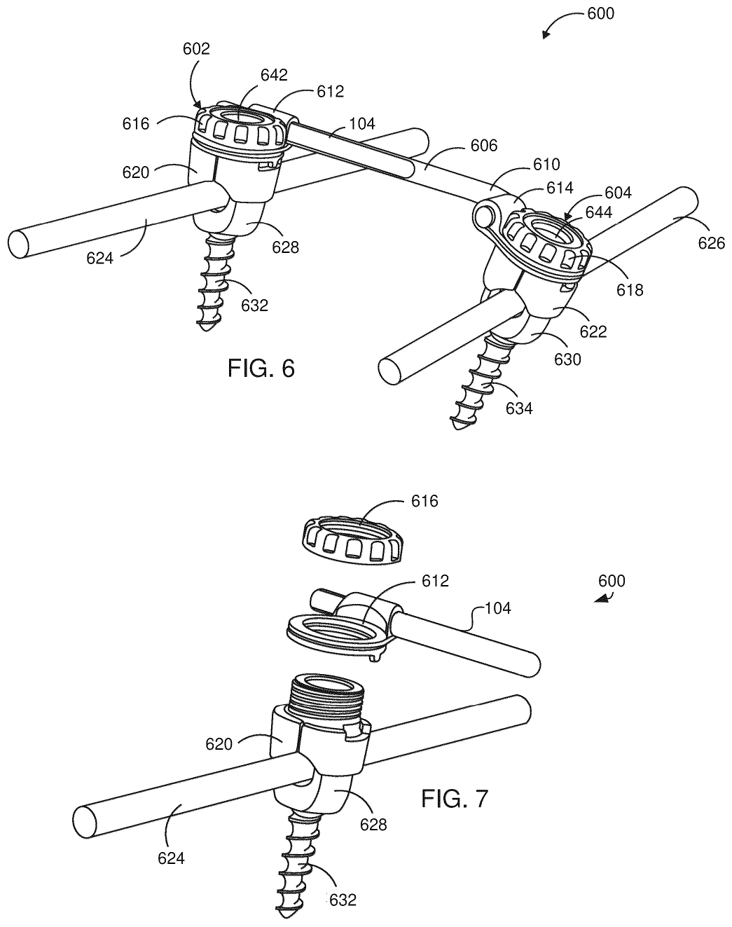

FIG. 6 illustrates an example implant 600 in the form of a spinal cross-connector 200 being a cartridge 104 or a cartridge 104 being disposed within a spinal cross connector 606. The implant includes an elongated cross connector 606, a first connector 602 and a second connector 604. The first connector 602 includes a first collet head 620 configured to receive a first tulip 628 and to accommodate the first spinal rod 624. Similarly, the second connector 604 includes a second collet head 622 configured to receive a second tulip 630 and to accommodate the second spinal rod 626. The first connector 602 and the second connector 604 are configured to receive the spinal rods 624 and 626 and directly attach with pedicle screws 632, 634 respectively. The cross connector 606 can include a flat portion that allows preventing the cross connector 606 from turning to three-hundred-and-sixty degrees thereby restricting the range of motion to a useful range. A first clamp 212 and a second clamp 214 can allow the cross connector 206 to translate for adjusting to the distance between the spinal rods 624 and 626. The first and second clamps 612, 614 get depressed by first and second locks 616 and 618 to lock the cross connector 606 in place. The first locking means 616 and the second locking means 618 can be tightened over a first collet head 620 and a second collet head 622 respectively. Upon tightening, first and second connectors 602, 604 get locked with the first and second tulips 628, 630 and the first and the second clamps 612, 614 get locked with the first and second ends 608, 610 of the elongated member 206 respectively.

FIG. 7 illustrates the example implant 600 in an exploded view. In addition, FIG. 7 illustrates that the implant 600 can be configured such that the cartridge 104 need not be used in a cross-connector fashion. The cartridge 104 can be cantilevered. In this configuration, the angle of the cartridge 104 can be adjusted prior to locking with the second locks 618.

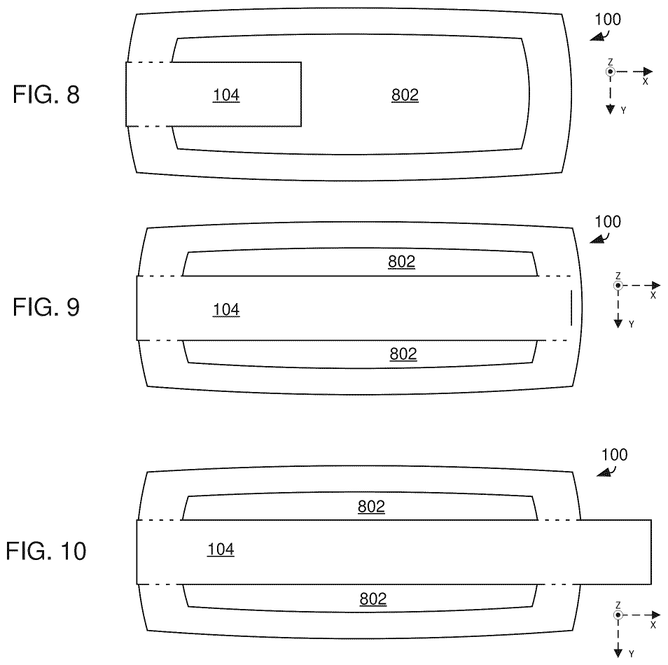

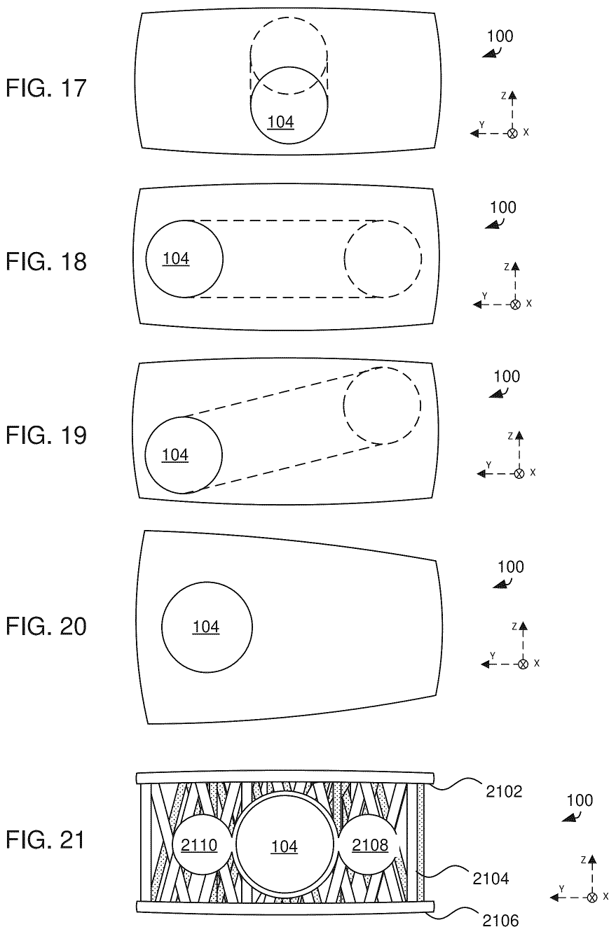

FIGS. 8-16 illustrates a cartridge 104 disposed in relation to an example intervertebral implant 100 as viewed from above. In these examples, the intervertebral implant 100 has a width extending in the Y direction, a length extending in the X direction, and a thickness in a Z direction. The implant 100 further defines a fusion aperture 802. The cartridge 104 can extend into or through the fusion aperture 802 in any of a variety of ways. These figures do not show positioning of the cartridge 104 in the Z dimension (examples of positioning the cartridge 104 with respect to the Z axis is shown in FIGS. 17-21). Thus, the cartridge 104 may be dimensioned such that fusion aperture 820 is not entirely blocked by the cartridge 104 (e.g., the fusion aperture 820 can extend above and/or below the cartridge 104).

A fusion aperture 802, apertures 802 extending between the upper and lower vertebral bodies which allow a boney bridge to form through the spinal fusion implant 100.

FIGS. 8-10 illustrate the cartridge 104 extending longitudinally with respect to the implant 100. The length of the cartridge 104 extends along the length of the implant 100 and the width of the cartridge 104 extends along the width of the implant 100. The cartridge 104 extends into the fusion aperture 802 from a short side of the implant 100. In FIG. 8, the cartridge 104 is cantilevered into the fusion aperture 802. In FIG. 9 the cartridge 104 extends entirely through the fusion aperture 802. In FIG. 10, the cartridge 104 extends entirely through the fusion aperture and out another side of the implant 100.

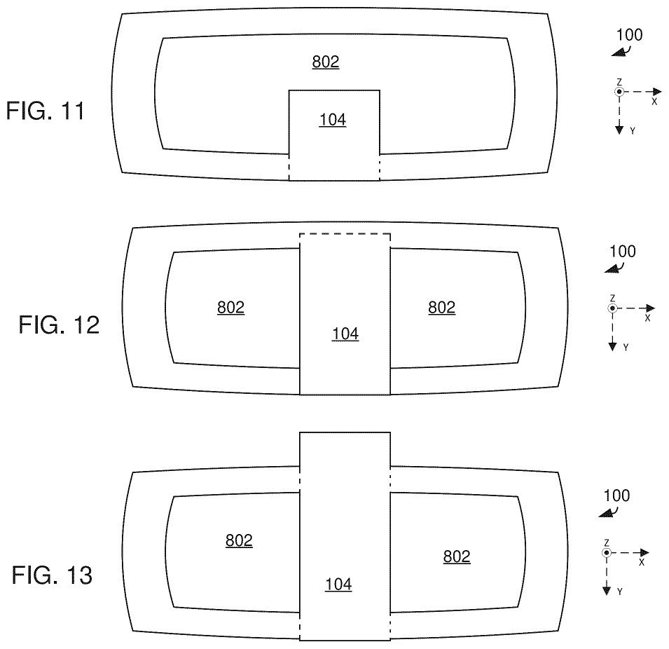

FIGS. 11-13 illustrate the cartridge 104 extending transversely with respect to the implant 100. The length of the cartridge 104 extends along the width of the implant 100 and the width of the cartridge 104 extends along the length of the implant 100. The cartridge 104 extends into the fusion aperture 802 from a long side of the implant 100. In FIG. 11, the cartridge 104 is cantilevered into the fusion aperture 802. In FIG. 12 the cartridge 104 extends entirely through the fusion aperture 802. In FIG. 13, the cartridge 104 extends entirely through the fusion aperture and out another side of the implant 100.

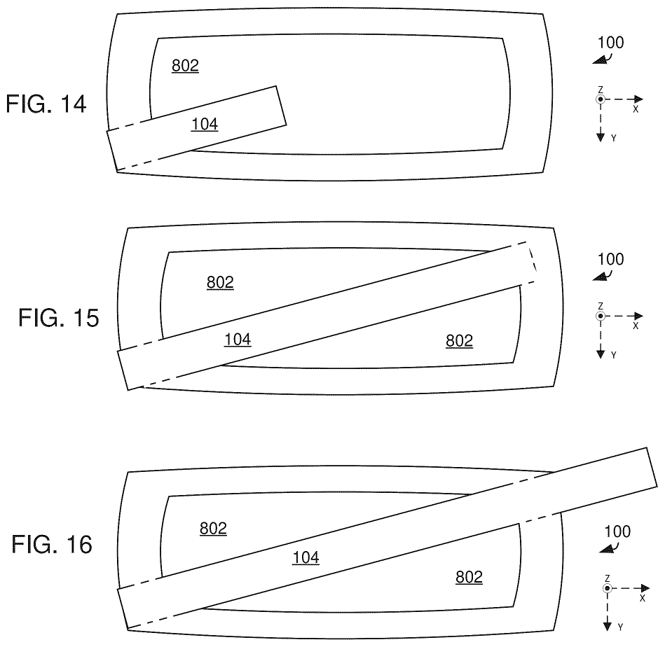

FIGS. 14-16 illustrate the cartridge 104 extending obliquely with respect to the implant 100 (e.g., neither perpendicular nor parallel to the length or width of the implant 100). The length of the cartridge 104 extends along both the width and length of the implant 100. The width of the cartridge 104 extends along both the width and length of the implant 100. As illustrated, the cartridge 104 extends at approximately a forty-five degree angle with respect to the length of the implant 100, but in other implementations, the cartridge 104 can be angled n-degrees with respect to the length of the implant 100, where 0<n<90 degrees. The cartridge 104 can extend into the fusion aperture 802 from one or both of a long side and a short side of the implant 100. In FIG. 14, the cartridge 104 is cantilevered into the fusion aperture 802. In FIG. 15 the cartridge 104 extends entirely through the fusion aperture 802. In FIG. 16, the cartridge 104 extends entirely through the fusion aperture and out another side of the implant 100.

FIGS. 17-21 illustrates a cartridge 104 disposed in relation to an example intervertebral implant 100 as viewed from a short side of the implant 100. As illustrated, the cartridge 104 can be disposed perpendicular to an edge of the implant 100. The cartridge 104 can be disposed parallel to the length of the implant 100. But as illustrated, the cartridge can be disposed obliquely with respect to the long axis of the implant.

FIG. 17 illustrates the cartridge 104 being non-parallel to the length of the implant, such as the relative position with respect to the height of the implant 100 varies along the length of the cartridge 104. In other words, points along the longitudinal axis of the cartridge vary in the z axis.

FIG. 18 illustrates the cartridge 104 being non-parallel to the length of the implant, such as the relative position with respect to the width of the implant 100 varies along the length of the cartridge 104. In other words, points along the longitudinal axis of the cartridge vary in the y axis.

FIG. 19 illustrates the cartridge 104 being non-parallel to the length of the implant, such as the relative position with respect to the width and height of the implant 100 varies along the length of the cartridge 104. In other words, points along the longitudinal axis of the cartridge vary in both the y axis and z axis.

FIG. 20 illustrates the position of the cartridge 104 being located near a portion of the implant 100 having a greater height. Spinal implants 100 can be wedge shaped to impart desired changes to a patient's spine (e.g., modify lordosis). It can be beneficial to have the implant 100 configured to receive the cartridge 104 in a position closer to a taller portion of the implant 100 than a shorter portion of the implant 100.

FIG. 21 illustrates an implant 100 having a cartridge 104 floating between a first endplate 2102 and a second endplate 2106 separated by struts 2104. The cartridge 104 can be considered floating in that the cartridge 104 does not contact either of the endplates 2102. The cartridge can be considered floating in that there are struts 2104 (or portions of struts 2104) that separate the cartridge 104 from the endplates 2102, 2106. As illustrated, inserter connectors 2108, 2110 are also floating with respect to the implant. The inserter connectors 2108, 2110 can be portions of the implant 100 configured to couple with an inserter instrument to facilitate implantation of the implant 100.

### Cartridge Configuration

For simplicity, the cartridges 104 illustrated in FIGS. 2-21 have been cylindrical or pill-shaped, but cartridges 104 can take any of a variety of forms.

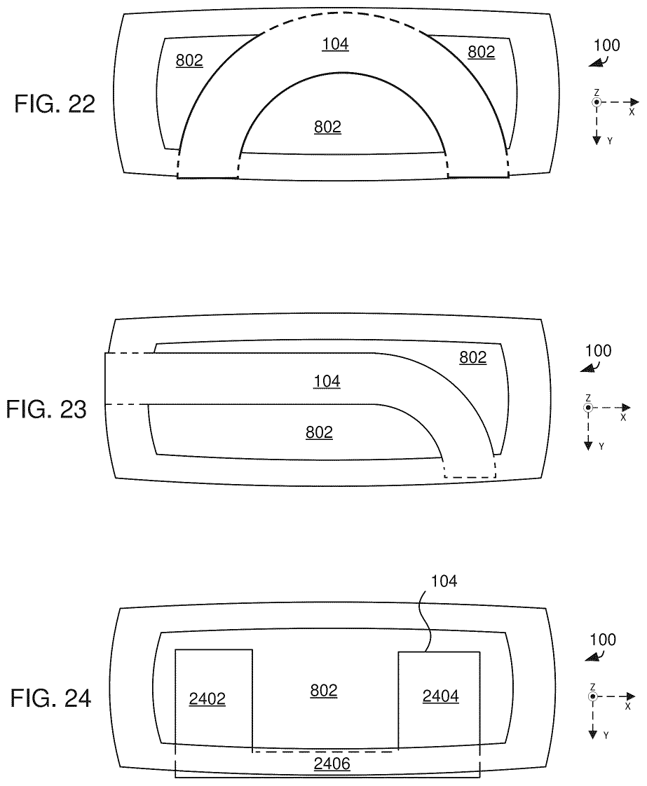

FIG. 22 illustrates a top-down view of an arcuate cartridge 104 disposed within an implant 100.

FIG. 23 illustrates a top-down view of an elongate cartridge 104 with an arcuate portion disposed within an implant 100.

FIG. 24 illustrates a top-down view of an implant 100 having an elongate cartridge 104 with first and second lobes 2402, 2404 separated by a connector 2406. Different components of the cartridge 104 can be placed in different lobes 2402, 2404. For example, the first lobe 2402 can contain the power source, the second lobe 2404 can contain the sensors 110, processors 130 and memory 140, and the connector can include the interface 150 (e.g., an antenna).

### Sensing

In an example, there can be multiple implants 100 configured such that sensor data between the implants 100 can be compared. For example, there can be spatial awareness between the implants 100. For example, angular differences between the implants can be determined. There can be a mesh network among multiple implants 100 where they are communicating with each other. Impedance between sensors 100 can be used to identify impedance changes to determine if fusion is occurring. There can be a paradigm to understand fusion. Having more than one implant 100 with sensors 110 (e.g., inertial measurement unit(s)) can be used to fuse that data together to determine localized general positions. This information can capture spine and human movement from gross motor movements. From that data, a world coordinate space within the human body can be determined. For example, using the data from the sensors 110, data can be processed such that vectors can be combined to understand that the body has moved by taking the fused data from two sensors. This provides a more robust paradigm than individual sensors. A sensor 110 can measure on, for example, at least three axes. If there are two or more datasets of micro-motion, the system 10 can fuse those to connect the vectors to better understand translation and rotation. In spine surgery, there are often many implants 100 being placed. One implant 100 can be a hub implant that has the bulk of the processing, etc. Smaller units can be used that don't have as much processing power feeding into that to do the communication outside. Further, meshing implants 100 together can be used to transmit data from a location where there is less attenuation from tissue (e.g., transmitting from a location closer to the patient's skin). The sensors 110 can sense load and tie in locational markers within the vertebra to indicate change in curvature or other properties. One or more implants 100 or cartridges 104 thereof can be an implanted hub that collects data from other sensors and then sends out that data so that curve progression or measurements can be made. In an example, one or more of the sensors 110 can be in the form of pressure sensitive films can be used and monitored to determine pressure. In an example, the sensors 110 can be used to detect screw backout. There can be a determination of the correlation between screw tightness and other factors. In an example, there can be a sensor 110 with contact electrification (e.g., as a layer in or on the implant 100). The sensor 110 can sense screw pressure pulling the plate down or the backing off over time as well. In an example, a sensor 110 can be a silver chloride electrode that is put on a nerve root to characterize whether pain exists or not. Can be used to determine if fusion is successful and for other purposes. In an example, a sensor 110 is in a pressure sensing load path to endplates of the implant 100. In an example, there is a cartridge 104 with dovetail connections to endplates that are being loaded such that at least some of the load is through the cartridge 104. In an example, RFID is used as a beacon for absolute position. To compensate for poor RFID tolerances, output from another sensor 110 can be used for dead reckoning to determine position. From there, positioning changes can be checked to determine movement and send an alert in response thereto. Sensors 110 can be used to monitor activities of spinal motion preservation devices (e.g., bumpers) and can be used to see how devices change the loading pattern on the spine. The spinous process bumpers or vertebroplasty devices where there are injected materials in (e.g., a material that has a viscoelastic characteristic that can be measured). In an example, the sensors 110 can be used to determine whether subsidence of the implant 100 has occurred based on changes in position. In an example, the sensors 110 can be used to determine proximal junctional kyphosis.

Any time an implant is placed in a patient, there is a concern for developing infection. Accordingly, one type of sensor that can be included in any of the smart implants discussed herein may include a biofilm detection sensor. In one embodiment, the sensor includes an RF transmitter, an RC receiver spaced apart from the RF transmitter (both the transmitter antenna and receiver antenna are typically disposed along a surface of the implant to be in contact with tissue), and circuitry that detects received signals and analyzes the signals for such attributes as an attenuation in a selected time period to determine the presence of any bacteria such as Staphylococccus aureus, Staphylococcus epidermidis, Streptococcus and the like. In one embodiment, the characteristic of attenuation of the RF signal is determined based on a reflected power of the RF signal provided by the circuitry toward the RF transmitter antenna. For example, the circuitry compares the characteristic of attenuation of the RF signal to a threshold characteristic of attenuation defined based on a calibration measurement operation and determines the presence of biofilm if difference beyond a set threshold value. In one way, the circuitry in the implant generates a range of RF signals sweeping through a defined frequency range and/or a defined amplitude range and detects the presence of biofilm based on identifying at least a threshold change in amplitude and/or frequency of the RF signals at one or more defined marker frequencies and/or defined marker amplitudes indicative of presence of a biofilm. In another embodiment, a temperature sensor in the implant can work in conjunction with the biofilm sensor to make a more accurate diagnosis of an infection. The circuitry monitors the temperature around the implant through the temperature sensor for a rapid rise in temperature within a select time period or an absolute temperature level. If the temperature change or the absolute temperature goes beyond a threshold value, the circuitry determines that an infection is likely. The circuitry may combine the readings and anlysis from the temperature sensor and the biofilm sensor to report an infection likelihood if both sensors indicate an infection.

In an example, stresses on the implant can be measured and then an alert can be sent to the patient that the position they are in or the activity (e.g., lifting something heavy) that they are engaging in is problematic. For instance, it can be problematic in a way that is likely to decrease the likelihood of successful spinal fusion. Or it can be problematic in a way that likely to increase subsidence of the implant 100. Or it can be problematic in a way that is likely to increase the risk of pedicle screw pullout or rod fracture.

In an example, the data from the sensors 110 is compared with wearable device data that is captured preoperatively or post-operatively.

The data from the sensors 110 can be used not just for monitoring fusion or loading, but can be used to connect to another device to measure or respond to patient's pain (e.g., can connect to a pain pump). Example other devices include activity monitors (e.g., FITBIT or APPLE WATCH devices). The sensors 110 can determine or inform patient satisfaction, which suggests restoration of activities and reduction of pain. The system 10 can further monitor how many pills someone takes or their activity level. Interventional care providers (e.g., pain docs) can benefit from and monitor that data. The output from the sensors 110 can be used to determine a pain profile and respond. Examples can include changing mechanical properties of an implant or providing electrical stimulation with the implant or an external device. Beneficially, incorporating the implant data into determining pain can provide objective measures. Further, the data can be used to determine a correlation between activity level or other behavior and pain.

Joint balancing can be useful to measure. The implant 100 can be an expandable implant, and the sensors 110 can be used to directly or indirectly measure force feedback and measure that for multiple levels intraoperatively. A user can implant an implant 100 and adjust the force and position. The sensors 110 can be monitored wirelessly or with leads coming out. With multiple implants 100, the user can have force feedback and on multiple levels and can see the correction that can continuously monitor during surgery to understand a force profile and how correction on one level affects other levels. There can be both global forces and local forces on an implant 100. Force monitoring can be discrete or continuous. There can be a feedback response loop at an adjacent level and can pick up modulation or other data and can automatically tension posterior fixation elements.

In an example, the implant 100 can store information regarding the implant 100 (e.g., device identifier) usable revision. For example, RFID can be used for storing the information. For instance, RFID can be placed in cannulated screws and measurable with NFC. There can be an associated chip and can flash changes to the chip and can change the ability to change the protocol for the transmit/receive the data on the RFID, so the data can be changed.

While electronic sensors have been discussed, non-electronic sensors can be used in addition or instead. For example, there can be an air pocket with low density in a material. When it is scanned after a period of time, a clinician can see how the implant has infiltrated or moved around. Cross modality detection can be useful. For example, the implant can have multiple different fiducials, each configured for different imaging modalities (e.g., radiopaque to MRI, ultrasound, etc.) For example, there can be a saline pocket in the middle of the implant that is really easy to detect in MR. In another example, there can be a layer of gel or another material that has acoustic properties change based on pressure. The changes in pressure can then be detected externally using another device, such as an ultrasound imaging device. In another example, there may be a mechanic ramp or other component that can change based on pressure or other mechanical forces. The mechanical component can be visualized using x-ray or other imaging modalities to sense properties.

### Implant

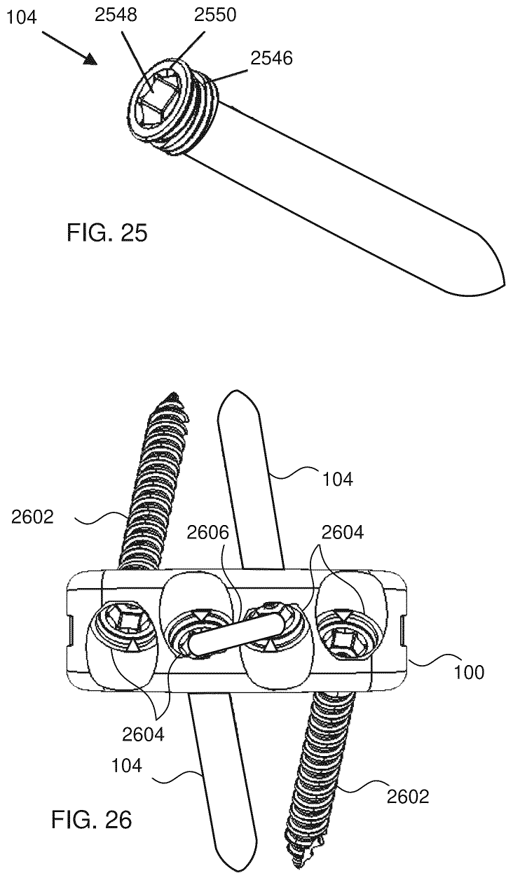

FIG. 25 illustrates an example cartridge 104 configured for insertion into a screw hole of an implant. The cartridge 104 as illustrated includes a tooling recess 2548 to facilitate insertion of the cartridge into a screw hole of an implant 100. The cartridge 104 can include a rim 2550 that can interact with a fixation feature (e.g., a screw backout feature) of the implant 100. The cartridge 104 includes a neck 2546. As illustrated, the neck can include threads that can cooperate or thread into an implant 100. In an example, there can be one or more electrical connectors in the tooling recess 2548 that can be used to connect to another cartridge 104 or device.

FIG. 26 illustrates an example anterior lumbar interbody fusion implant 100 with bone screws 2602 and two cartridges 104 in the form illustrated in FIG. 25 inserted. In such a manner, the implant 100 can be inserted (e.g., via an ALIF approach). The illustrated implant 100 includes four fixation apertures 2604 (though other numbers can be used) through which screws are traditionally inserted. As illustrated, only two of the fixation apertures 2604 are used to hold bone screws 2602 (e.g., that extend into a vertebral body). The middle two fixation apertures 2604 are taken up by cartridges 104. The cartridges 104 when inserted in this manner, enter superior or inferior endplates and into respective vertebral bodies. As illustrated, there is also a connector 2606 that connects the two cartridges 104 so they can cooperate.

In an example method, a spinal implant 100 is implanted between a superior vertebra and an inferior bone (e.g., a vertebra or sacrum), such as via an anterior access approach. After insertion, one or more bone fixators (e.g., bone screws or blades) are inserted into one or more fixation apertures 2604 of the implant 100 and into one or both of the superior vertebra and inferior bone. After insertion of the implant 100, one or more cartridges 104 are inserted into the spinal implant 100 and into one or both of the superior vertebra or inferior bone (e.g., through a vertebral endplate and into a vertebral body). In an example, the cartridge 104 includes one or more structures (e.g., threads, blades, cutting tip) that facilitate its insertion into bone. In some examples, a pilot hole is drilled or another device is used to make room for the cartridge 104 in the bone prior to insertion. In an example, the cartridge 104 is coupled to another device via a connector 2606. For example, there can be multiple cartridges inserted into the implant 100 (and into bone or merely into the implant 100) and the cartridges are electrically connected via a physical connector 2606.

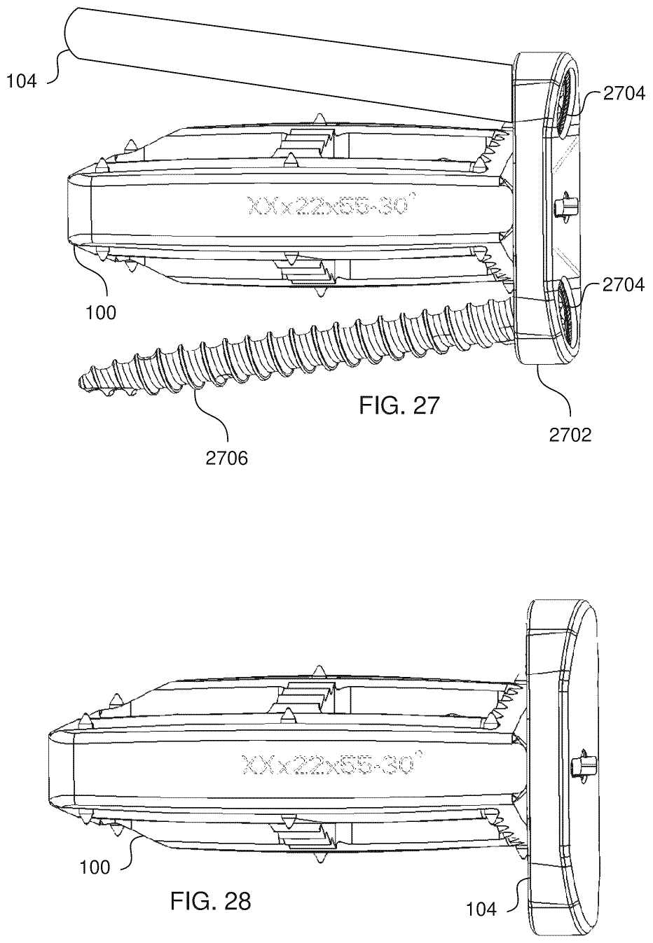

FIG. 27 illustrates an example implant 100 with a plate 2702. The plate 2702 is coupled to the implant or integral with the implant 100. As illustrated, the plate 2702 is substantially perpendicular to the length of the implant 100. As illustrated, the plate 2702 includes two fixation apertures 2704. Traditionally, such plates 2702 include one or two fixation apertures to facilitate holding the implant 100 in place with a bone screw 2706 (see, e.g., U.S. Pat. No. 10,842,642, filed Jun. 21, 2018 as application Ser. No. 16/015,182, which is hereby incorporated herein by reference in its entirety for any and all purposes). However, as illustrated here, in place of one of the bone screws 2706 is a cartridge 104, such as is described in FIG. 25.

In an example method, a spinal implant 100 is implanted between a superior vertebra and an inferior bone (e.g., a vertebra or sacrum), such as via an anterior access approach. The implant 100 includes a built in plate 2702 or, in a step of the method, the plate 2702 is coupled to the implant 100 (e.g., after implantation of the implant 100 or prior to implantation of the implant 100). One or more bone fixators (e.g., bone screws or blades) are inserted into one or more fixation apertures 2704 of the plate 270 and into one or both of the superior vertebra and inferior bone. One or more cartridges 104 are inserted into the plate 2702 and into one or both of the superior vertebra or inferior bone (e.g., a vertebral body). In an example, the cartridge 104 includes one or more structures (e.g., threads, blades, cutting tip) that facilitate its insertion into bone. In some examples, a pilot hole is drilled or another device is used to make room for the cartridge 104 in the bone prior to insertion. In an example, the cartridge 104 is coupled to another device via a connector 2606. For example, there can be multiple cartridges inserted into the implant 100 (and into bone or merely into the implant 100) and the cartridges are electrically connected via a physical connector 2606.

FIG. 28 is an example implant 100 with a cartridge 104 coupled thereto in the form of a plate (e g, similar to plate 2702 with or without fixation apertures). As illustrated, the cartridge 104 has a length that is substantially perpendicular to the length of the implant 100. Although as illustrated, the cartridge 104 is entirely contained in the plate, in addition or instead, the cartridge 104 or components thereof is included in the implant 100. For example, one or more sensors can be disposed in the body of the implant 1000 and an antenna or battery components are disposed in the plate portion. In an example, the plate is constructed form a different material than the implant 100. For example, the body of the implant 100 can be constructed from titanium and the plate is constructed from PEEK.

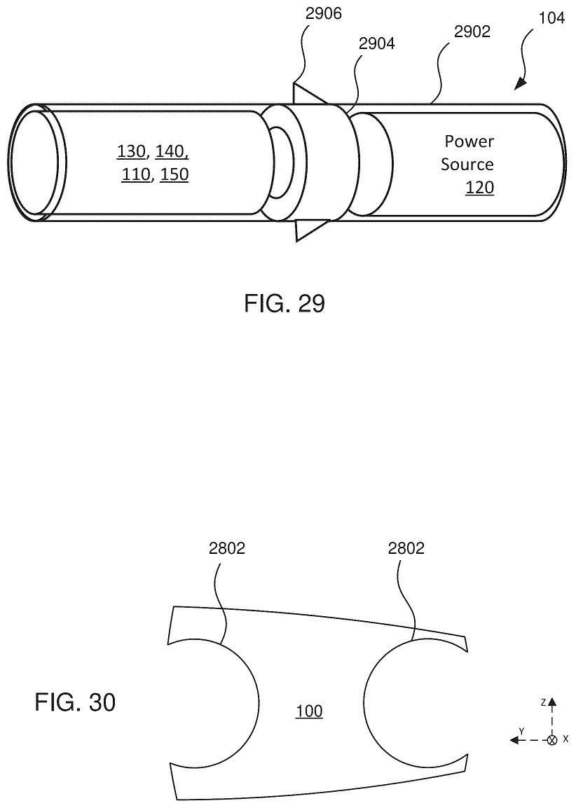

FIG. 29 illustrates a partial cutaway view of a cartridge 104. The cartridge 104 includes a housing 2902 that surrounds components, such as one or more sensors 110, one or more processors 130, one or more power sources 120, one or more memory unit 140, one or more interfaces 150. In addition, the illustrated cartridge 104 includes one or more strengtheners 2904. As illustrated, the strengthener 2904 is a disc of thick material, but can take other forms, such as one or more struts, structures, or other strengtheners. The strengthener 2904 can be configured to increase the strength of the cartridge 104 at the location of the strengthener 2904. The cartridge 104 can include a housing 2904, but the strength profile of the cartridge 104 can vary along the length. The strengthener 2904 can increase the strength in a particular region of the cartridge 104, such as to protect one or more components. The strengthener 2904 can be a location by which one or more connectors 2906 are coupled to the cartridge 104. For example, the connectors 2906 can be locations or features by which the cartridge 104 is coupled to the implant 100. In an example, one or more cartridges can be or cooperate with locking tapers. In some examples, the strengthener 2904 can be a region at which compressive forces are directed through the cartridge 104 (e.g., as opposed to other, more vulnerable regions). The strengthener 2904 can be a region at which a strain or stress sensor is located. The presence of the strengthen 2904 can permit regions of the cartage 104 to be constructed from weaker, radiopaque material without compromising the overall strength of the cartridge 104. The strengthener 2904 can be the only or the only substantial loadbearing component. Although the cartridge 104 is illustrated as having one strengthener 2904 located medially, there can be more than one strengthener 2904 in various locations. In an example, the strengthener 2904 can be configured to provide strength surrounding components sensitive to compression (e.g., certain power sources 120). In an example, certain portions of the cartridge 104 can be configured such that regions of the cartridge 104 are permitted to be weaker than others. The weaker portions can be portions surrounding or near components not sensitive to pressure, such as certain implementations of antennas.

FIG. 30 illustrates an example implant 100 in a lateral view having anterior and posterior cutouts 3002 in anterior and posterior faces of the implant 100 configured to receive one or more cartridges 104. In example, the superior and inferior endplates of the implant 100 form overhangs over the cutouts 3002. The cutouts 3002 can be configured such that cartridges 104 can snap into place. There can be separate anterior and posterior cartridges 104 inserted into the implant and then optionally directly or indirectly coupled.

### Implant-Cartridge Packaging

In an example, the body of the implant 100 can be manufactured from multiple different materials. There can be a 3D printed titanium implant and injection molding a plastic (e.g., PEEK) enclosure that couples to an anterior or posterior aspect of the implant to change at least one dimension of the implant to accommodate room for the cartridge 104. There can be axial loading structures. The plastic aspects can facilitate antenna communication. There can be posterior and anterior sides with coupling between to go ahead and do a coating (e.g., PVC) of the entire interbody so there can be a connector between the two (e.g., via wire, flex PCBA, etc.). If the PCBA can be a thin part and can be placed into a small package and the battery and antenna can be placed in relatively larger areas. There can be a T-shaped enclosure where the antenna, etc. are in the narrower part of the interbody.

In an example, the implant 100 can be constructed (e.g., molded or 3D printed) to either shield or load the cartridge 104 from/with stress. In an example, the implant 100 can be constructed to enhance or amplify signal being sent to or from the cartridge 104. In addition or instead, the implant 100 can be configured to block or inhibit signals that could cause interference.

In an example, the implant 100 can be partially or entirely formed by 3D printing implant structure around the cartridge 104. For example, the implant 100 can be partially printed, then the cartridge 104 is placed inside, and then the printing is finished around the cartridge 104. This printing can create a seal around the cartridge 104.

In an example, the implant 100 is manufactured in multiple pieces and then assembled together with the cartridge 104. For example, the implant 100 is 3D printed in two halves that are then assembled together by hand. The portions can come together in a way that supports the structure to encourage integrity of the implant 100.

The cartridge 104 may but need not be hermetically sealed in the implant 100. In an example, only part of the implant 100 is hermetically sealed. For example, the power source 120 can be hermetically sealed, but other components might not be so sealed. In an example implementation, there can be an antenna that is not hermetically sealed. There can be a stenciled inverted-F antenna. There can be a coating (e.g., PVC) of the board and the PCBA (Printed Circuit Board Assembly) is hermetically sealed. This can reduce removes risk and complexity of the enclosure of the cartridge 104.

The cartridge 104 or a PCBA thereof can be disposed in a distal nose of the implant 100. Instead of burying the whole package in the implant 100, part of the implant 100 can be replaced with the cartridge 104 (e.g., forming a solid endcap). There can be one or more tabs coming off of the cartridge 104 and into the implant 100 to keep the cartridge-implant assembly stable. There can be spring latch features that click onto the implant 100 (or into the cartridge). There can be an undercut with a built in snap fit that pops the components together. There can be a horizontal taper for holding the components together. There can a non-curved locking taper.

Where the cartridge 104 is inserted into the implant 100, there can be some play within the free space that surrounds the cartridge, which can reduce stress loading of the cartridge 104. In other examples, the cartridge can be held tight in the implant 100.

The cartridge 104 can have a circular cross section, such that the cartridge 104 can land in any orientation (e.g., can be rotated 360 degrees). There can be a calibration process for the cartridge 104 to zero its position or otherwise take into account its position for later sensing of data. The cartridge 104 can but need not be indexed. There can be a ground reaction plane that's a vector in the world that's coming straight down. Then, based on output from inertial measurement unit or other sensor, orientation relative to the ground reaction plane can be determined. There can be offset correction so the algorithm automatically offsets the initial starting position of the accelerometer (or other sensor data) and matches it to perpendicular or otherwise normalizes the data. The cartridge 104 can include a magnetometer. In an example, a calibration process can be performed post-operatively. For instance, data can be measured while the patient is in a known position (e.g., standing upright and facing North), and then subsequent data measurements can be compared to data measured in the known position. In an example, the calibration occurs in an operating room. For example, during assembly of the cartridge 104 into the implant 100, the assembly of the two can be put into a loading fixture or other calibration device to learn the position prior to implantation. In an example, the cartridge 104 can include a radiopaque marker or other identifiable marking to determine the orientation of the cartridge 104.

### Fixation System

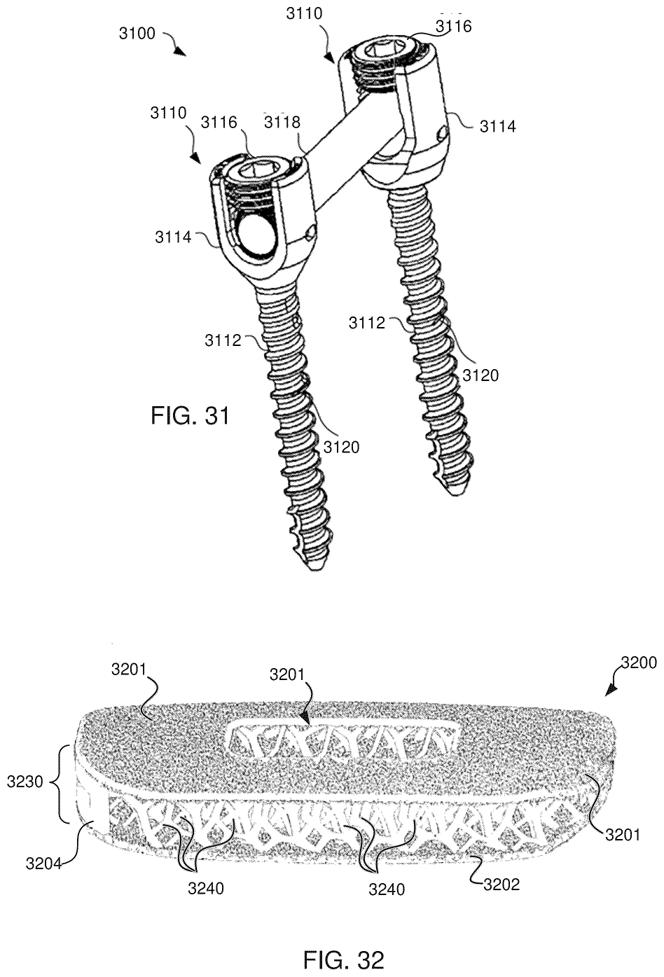

FIG. 31 illustrates a spinal fixation system 3100 benefitting from aspects disclosed herein. Certain of the fixation system 3100 components are based on those described in U.S. Pat. No. 7,833,251 (app. Ser. No. 11/031,506), filed Jan. 6, 2005, which is incorporated herein by reference in its entirety for any and all purposes, and include pedicle screw assemblies 3110 and a rod 3118. The pedicle screw assemblies 3110 each include a screw 3112, a tulip 3114, and a setscrew 3116. The screw 3112 can be a pedicle screw integral with or couplable to the tulip 3114. The tulip 3114 is a component configured to receive the rod 3118, which is held in place by the setscrew 3116

### Static Interbody Implant

FIG. 32 illustrates an example lateral spinal fusion implant 3200, which is described in more detail in US 2020/261243, which is hereby incorporated herein by reference in its entirety for any and all purposes. The implant 3200 has upper and lower endplates 3201, 3202 formed of a microporous endplate structure 3210 and a central body portion 3230 formed of a body lattice structure 3220. The implant 3200 has a leading end 3270 and an opposite trailing end 3280, and a fusion aperture 3203 extending through the implant 3200 from the upper bone contacting surface 3201 to the lower bone contacting surface 3202. The trailing end 3280 includes an instrument engagement feature 3204 that includes at least one engagement portion(s) 3205 for the engagement of an insertion tool. The leading end 3270 may be tapered to facilitate insertion into the disc space. In an alternative embodiment, at least a portion of the leading end 3270 is solid. According to this exemplary embodiment, the length dimension of the implant 3200 from leading end 3270 to trailing end 3280 is in the range from 45 mm to 65 mm, the anterior to posterior width dimension of the implant 3200 is in the range of 18 mm to 26 mm and angle of lordosis is in the range of 0° to 15°. It is also contemplated that the implant 3200 of present disclosure may have a hyperlordotic angle of lordosis ranging from 15° to 40°. The implant 3200 can be formed by a 3D printing process.

The upper and lower endplates 3201 and 3202 are formed of microporous endplate structure 3210 with a pore size, pore volume, strut 3240 thickness, and surface roughness design to promote bone growth and elicit an osteogenic response at the implantation site. According to one exemplary embodiment, the pores in the microporous endplate 3210 range in diameter from 3200 μm to 1500 μm, and the strut 3240 thicknesses ranges from 100 μm to 500 μm. In some embodiments, the pores in the microporous endplate 3210 range in size from 300 μm to 1200 μm and the strut 3240 thicknesses range in size from 150 μm to 300 μm. In one exemplary embodiment, the average pore diameter is 500 μm and the average strut 3240 thickness is 200 μm. According to an alternative embodiment, the average pore diameter is 800 μm and the average strut 3240 thickness is 200 μm. According to another exemplary embodiment, the microporous endplate structure 3210 forming the upper and lower contact surfaces 3201, 3202 have an average pore diameter of 500 μm at the perimeter and transitions to an average pore diameter of 800 μm toward the center of the upper and lower bone contacting surfaces 3201, 3202. The transition may be gradual or discrete. According to these exemplary embodiments, the microporous endplates 3201, 3202 have a macro surface roughness comprising protrusions extending up to 300 pin from the endplate surface and a nano/micro surface roughness comprising a surface texture ranging in depth from 0.45 μm to 7 μm.

The spinal fusion implant 3200 according to the embodiment further includes an implant frame 3290. The frame 3290 may comprise a solid rim bordering the outer perimeter and inner perimeter of the upper and lower contact surfaces 3201, 3202. In this embodiment the solid rim along the interior of the upper and lower contact surfaces 3201, 3202 forms the boundary of the fusion aperture 103.

In some embodiments, the implant 3200 includes at least one radiopaque marker 200 in the medial plane of the implant 3200. In some embodiments, the implant 3200 includes at least two radiopaque markers 200 in the medial plane. It is further contemplated that the implant 3200 of this disclosure can be used in conjunction with a fixation plate that is coupled to the trailing end 3280 of the implant 3200 and includes at least one fixation aperture for receiving a fixation element therethrough, such that the fixation aperture lies adjacent the lateral aspect of the vertebral body when the fixation plate is coupled to the implant 3200. In some embodiments, the fixation plate includes two fixation apertures, one that will lie adjacent to the lateral aspect of the superior vertebral body and one that will lie adjacent to the lateral aspect of the inferior vertebral body.

While this example implant focused on a lateral spinal implant having a strut structure, other implants can also benefit from techniques described herein. For example, there can be spinal implants having structures configured for use in anterior interbody fusion procedures, posterior interbody fusion procedures, transforaminal interbody fusion procedures, other procedures, or combinations thereof. The implants can be configured for use with specific anatomy, such as cervical, thoracic, or lumbar anatomy or patient-specific anatomy. Example anterior lumbar interbody fusion implants are described in U.S. Pat. Nos. 8,740,983 and 8,940,030, which are hereby incorporated herein by reference in their entirety for any and all purposes. Other interbody devices include those described in U.S. Pat. Nos. 7,815,682; 7,918,891; 8,187,334; 8,246,686; 8,287,597; 8,361,156; 8,574,301; 8,608,804; 8,673,005; 8,685,105; 8,814,940; 8,920,500; 9,180,021; 9,186,261; 9,192,482; D594,986; D599,019; D621,509; D671,645; D674,092; D675,320; D696,402; D708,747; D735,336; D747,485; D750,252; D754,346; D759,248; D767,762; D770,405; 9,474,627; 9,486,329; D781,423; 9,744,053; D788,307; D788,308; D791,949; D797,934, which are each incorporated herein by reference in their entirety for any and all purposes.

### Total Disc Replacement Implants

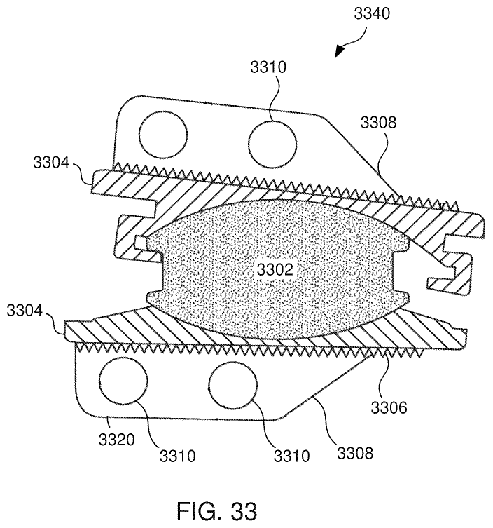

FIG. 33 illustrates a lateral partial cross sectional view of a prosthetic disc 3340 that can benefit from aspects disclosed herein. Certain features of the prosthetic disc 3340 are based on those described in U.S. Pat. No. 9,107,762 (app. Ser. No. 13/830,028), filed Nov. 3, 2011, which is incorporated herein by reference in its entirety for any and all purposes, and includes a core 3302 disposed between plates 3304 having serrations 3306 and fins 3308 defining apertures 3310. The implant 3340 can benefit from the inclusion of one or more retention features 3320, such as teeth or fins to resist movement of the implant.

### Bone Plates

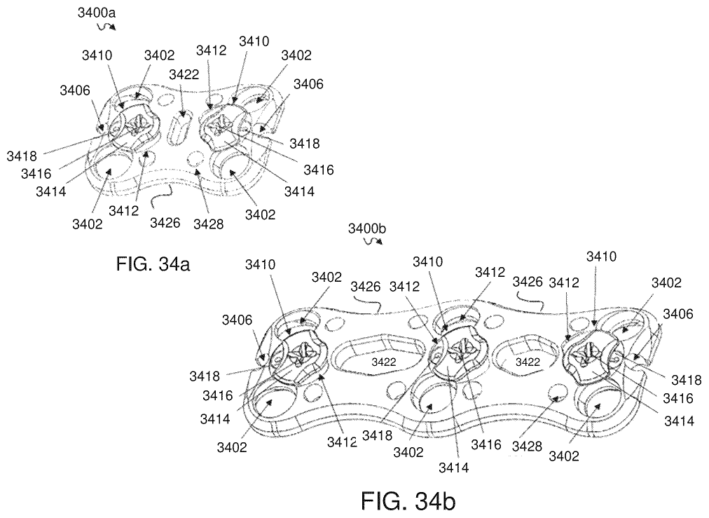

Technology described herein can be applied to bone plates, such as an anterior cervical plates, anterior lumbar plates, lateral plates, or other plates, FIGS. 34a and 34b show an example of anterior cervical plate. FIG. 34a shows a single-level bone plate 3400a and FIG. 34b shows a two-level bone plate 3400b. Single-level bone plate 3400a allows for fixation of two adjacent vertebrae whereas 2-level bone plate 3400b allows for fixation of three adjacent vertebrae. Features of the bone plates 3400a, 3400b are substantially identical, therefore, like numbering in both figures represent identical features and bone plates 3400a, 3400b will not be discussed individually.

The bone plate 3400a, 3400b includes a set of fixation apertures 3402. The fixation apertures 3402 enable the bone plate 3400a, 3400b to be fixed to vertebrae by insertion of bone screws or fasteners (not shown), such as, for example, fixed or variable bone screws therein. The fixation apertures 3402 are arranged in pairs of transversely aligned apertures. The number of pairs of fixation apertures 3402 may be dependent on the number of levels of desired spinal stabilization. While FIG. 11 shows a single-level bone plate 3400a and FIG. 12 shows a 2-level bone plate 3400b, a bone plate according to embodiments of the disclosure may also include a 3-level, 4-level, and 5-level bone plate. Each additional level plate will include an additional pair of bone fixation apertures and may or may not include a visualization aperture between sets of additional bone fixation apertures. The bone plate 3400a, 3400b may optionally include apertures 3406 at cranial and caudal ends of bone plate 3400a, 3400b for temporary tacks or temporary pins (not shown) for temporarily holding bone plate 3400a, 3400b to bone. Such apertures 3406 enable bone plate 3400a, 3400b, to be temporarily secured to the desired position relative to the desired vertebrae while bone plate 3400a, 3400b is fixated to the bone (not shown).

The bone plate 3400a, 3400b may also include an anti-backout mechanism 3410 positioned at each pair of fixation apertures 3402. Anti-backout mechanisms 3410 may be rotatably disposed within recesses 3412 formed within the anterior surface of bone plate 3400a, 3400b. The anti-backout mechanisms 3410 may include projections 3414 for at least partially covering bone screws that have been inserted into the fixation aperture 3402. In this way, anti-backout mechanisms 3410 retain bone screws within the fixation apertures 3402 once the bone plate 3400a, 3400b has been fixed to the bone. Anti-backout mechanisms 3410 may include a mating feature 3416 which can have a shape and/or configuration to allow operable engagement with a complementary feature on a locking instrument (not shown). The locking instrument may engage mating feature 3416 of the anti-backout mechanisms 3410 and be configured to rotate the anti-backout mechanisms 3410 to actuate the anti-backout mechanisms 3410 between an unlocked position and a locked position. When anti-backout mechanism 3410 is in the unlocked position, the fixation apertures 3402 are exposed to allow passage of a bone screw therethrough. After bone screws are inserted into the fixation apertures 3402, the anti-backout mechanism 3410 can be actuated to the locked position such that projections 3414 at least partially cover bone screws within fixation apertures 3402 thereby retaining the bone screws within the fixation apertures 3402.

The bone plate 3400a, 3400b may also include ring or clip member (hereinafter “ring member”) 3418 positioned within the recesses 3412 and beneath the anti-backout mechanisms 3410. The ring members 3418 may provide resistance to the anti-backout mechanisms 3410 thereby prohibiting the anti-backout mechanisms 3418 from rotating after surgery and/or without the locking instrument. Further, the ring members 3418 may provide tactile feedback to the user such that the user is able to discern the change in position of the anti-backout mechanism 3410 as the anti-backout mechanism 3410 transitions between the locked and unlocked position by the locking instrument. Specifically, each ring member 3418 may cause an increase in torque as the anti-backout mechanisms 3410 transition and/or rotate between the locked and unlocked position. As the anti-backout mechanism 3410 is rotated relative to the ring member 3418, a plurality of flat surfaces 3411a and peaks 3411b that separate the flat surfaces 3411 a cause the engagement member 3411 to interact with the ring member 3418. When the peaks 3411b interact with and/or engage the ring member 3418, the ring member 3418 flexes outward providing a torque delta. As the anti-backout mechanism 3410 continues to rotate about the ring member 3418, the ring member 3418 collapses against the flat surfaces 3411 a thereby holding the anti-backout mechanism 3410 in place.

Bone plates 3400a, 3400b may be provided having any number of different peripheral profiles, including but not limited to, the generally rectangular peripheral profiles shown in FIGS. 11-12. Bone plates 3400a, 3400b may optionally include viewing apertures 3422 positioned generally within the central portion of bone plate 3400a, 3400b and between pairs of fixation apertures 3402. Viewing apertures 3422 provide the ability to see or visualize the spinal target site after the bone plate 3400a, 3400b has been secured to the patient. It will be appreciated that viewing aperture 3422 may be provided in any number of suitable shapes or configurations without departing from the scope of the disclosure, and therefore is not limited to the shape shown by way of example in the Figures.

The bone plate 3400a, 3400b may also include indentations 3426 positioned along the lateral sides of bone plate 3400a, 3400b between each pair of fixation apertures 3402. Optionally, indentations may also be included on cranial and caudal ends of bone plate 3400a, 3400b. Indentions 3426 reduce the amount of material used in manufacturing bone plates 3400a, 3400b and reduce the overall profile of bone plate 3400a, 3400b. In addition, the bone plate 3400a, 3400b may include substantially rounded edges to further reduce the amount of material used in manufacturing and reduce the overall profile. The bone plates 3400a, 3400b may be of any desired thickness, such as, for example, 1.6 mm, 1.9 mm, or 2.1 mm. However, it is to be understood that other plate thicknesses are also contemplated.

Further, the bone plate 3400a, 3400b may include a plurality of insertion apertures 3428 arranged about bone plate 3400a, 3400b. Insertion apertures 3428 are configured to receive at least a portion of an insertion device (not shown) and may be used to aid the insertion of bone plate 3400a, 3400b in a desired position relative to the bone. Insertion apertures 3428 may include features complementary to the insertion device to facilitate engagement therewith, such as for example, threads.

### Other Implants

Various implants can benefit from aspects described herein, including spinous process implants (e.g., as described in U.S. Pat. Nos. 8,343,190; 7,842,074; 8,002,802; 8,292,923; D725,270, which are incorporated herein by reference in their entirety for any and all purposes), expandable interbody devices (e.g., as described in U.S. Pat. Nos. 9,801,734; and 9,445,918, which are hereby incorporated herein by reference in their entirety for any and all purposes), expandable or static vertebral body replacement devices (e.g., as described in U.S. Pat. Nos. 9,387,090; 9,687,357; and 9,636,233), total disc replacement implants (e.g., as described in U.S. Pat. Nos. 8,328,851; 8,870,960; 9,168,149; 10,441,431; 10,130,494; 10,085,853; 9,956,091; 9,839,532; 9,839,525; 9,439,775; 9,421,107; 9,107,762; 8,974,533; 8,845,729; 8,808,384; 8,764,833; 8,454,698; 8,444,695; 8,062,371; 8,002,834; 7,753,956; 7,585,326; 7,442,211, which are incorporated herein by reference in their entirety for any and all purposes), scoliosis correction devices (e.g., as described in U.S. Pat. Nos. 8,057,472; 8,197,490; 8,343,192; 8,382,756; 8,419,734; 8,715,159; 8,734,488; 8,852,236; 8,974,463; 9,011,499; 9,179,938; 9,179,960; 9,186,183; and 9,198,755, each of which are incorporated herein by reference in their entirety for any and all purposes).

Example techniques for implementing such computer functions include frameworks and technologies offering a full stack of plug-and-play capabilities for implementing desktop and browser-based applications (e.g., the applications implementing aspects described herein). The frameworks can provide a desktop web application featuring or using an HTTP server such as NODEJS or KATANA and an embeddable web browser control such as the CHROMIUM EMBEDDED FRAMEWORK or the JAVA/.NET CORE web view. The client-side frameworks can extend that concept by adding plug-and-play capabilities to desktop and the web shells for providing apps capable of running both on the desktop and as a web application. One or more components can be implemented using a set of OWIN (Open Web Interface for .NET) components built by MICROSOFT targeting the traditional .NET runtime. KATANA, and by definition OWIN, allow for chaining together middleware (OWIN-compliant modules) into a pipeline thus offering a modular approach to building web server middleware. For instance, the client-side frameworks can use a Katana pipeline featuring modules such as SIGNALR, security, an HTTP server itself. The plug-and-play capabilities can provide a framework allowing runtime assembly of apps from available plugins. An app built atop of a plug-and-play framework can have dozens of plugins, with some offering infrastructure-level functionality and other offering domain-specific functionality. The CHROMIUM EMBEDDED FRAMEWORK is an open source framework for embedding the CHROMIUM browser engine with bindings for different languages, such as C # or JAVA. OWIN is a standard for an interface between .NET web applications and web servers aiming at decoupling the relationship between ASP.NET applications and IIS by defining a standard interface.

Further example techniques for implementing such computer functions or algorithms include frameworks and technologies provided by or in conjunction with programming languages and associated libraries. For example, languages such as C, C++, C #, PYTHON, JAVA, JAVASCRIPT, RUST, assembly, HASKELL, other languages, or combinations thereof can be used. Such languages can include or be associated with one or more standard libraries or community provided libraries. Such libraries in the hands of someone skilled in the art can facilitate the creation of software based on descriptions herein, including the receiving, processing, providing, and presenting of data. Example libraries for PYTHON and C++ include OPENCV (e.g., which can be used to implement computer vision and image processing techniques), TENS ORFLOW (e.g., which can be used to implement machine learning and artificial intelligence techniques), and GTK (e.g., which can be used to implement user interface elements). Further examples include NUMPY for PYTHON (e.g., which can be used to implement data processing techniques). In addition, other software can provide application programming interfaces that can be interacted with to implement one or more aspects described herein. For example, an operating system for the computing environment (e.g., WINDOWS by MICROSOFT CORP., MACOS by APPLE INC., or a LINUX-based operating system such as UBUNTU by CANONICAL LTD.) or another component herein (e.g., an operating system of a robot, such as IIQKA.OS or SUNRISE.OS by KUKA ROBOTICS CORPORATION where the robot is a model of KUKA ROBOTICS CORPORATION) can provide application programming interfaces or libraries to usable to implement aspects described herein. As a further example, a provider of a navigation system, laser console, wireless card, display, motor, sensors, or another component may not only provide hardware components (e.g., sensor, a camera, wireless card, motor, or laser generator), but also software components (e.g., libraries, drivers, or applications) usable to implement features with respect to the components.

What is claimed is:

1. An intervertebral implant comprising:
a body adapted to be implanted into an intervertebral space of a patient, the body defining a fusion aperture;
at least one cartridge with each cartridge disposed in the body and at least partially extending into the fusion aperture, the each cartridge including:
an impedance sensor configured to measure electrical resistance;
a radio frequency (RF) transmit antenna configured to transmit a RF signal;
circuitry and operative to:
record data from the impedance sensor;
generate the RF signal containing the recorded data;
transmit the generated RF signal to an external clinician computing device through the RF transmit antenna.

2. The intervertebral implant of claim 1, wherein the body includes a through hole in communication with the fusion aperture and the cartridge is at least partially disposed in the through hole.

3. The intervertebral implant of claim 1, wherein the fusion aperture is an elongate aperture defining a central longitudinal axis, and the cartridge extends along the central longitudinal axis.

4. The intervertebral implant of claim 3, wherein the cartridge extends through the fusion aperture.

5. The intervertebral implant of claim 3, wherein the cartridge extends into the fusion aperture in a direction lateral to the central longitudinal axis.

6. The intervertebral implant of claim 1, wherein the cartridge includes a biofilm sensor for detecting the presence of biofilm, the biofilm sensor including a signal transmitter and signal receiver overlying an external surface of the cartridge.

7. The intervertebral implant of claim 6, wherein the cartridge includes circuitry for analyzing attenuation of a signal transmitted by the signal transmitter and received by the signal receiver.

8. The intervertebral implant of claim 7, wherein the circuitry is configured to generate a range of RF signals sweeping through a defined frequency range or amplitude range, and detect the presence of biofilm based on identifying at least a threshold change in amplitude or frequency of the received signal at one or more defined marker frequencies or defined marker amplitudes.

9. The intervertebral implant of claim 1, wherein the at least one cartridge includes a plurality of cartridges with each disposed in a respective body and that communicate with each other in a mesh network.

10. The intervertebral implant of claim 1, wherein each cartridge includes an inertial measurement unit and a load sensor.

11. An intervertebral implant comprising:
a body adapted to be implanted into an intervertebral space of a patient, the body defining a fusion aperture;
at least one cartridge with each cartridge disposed in the body and at least partially extending into the fusion aperture, the each cartridge including:
an impedance sensor configured to measure electrical resistance;
an inertial measure unit sensor for measuring an orientation of the implant;
a radio frequency (RF) transmit antenna configured to transmit a RF signal;
circuitry and operative to:
record data from the sensors;
generate the RF signal containing the recorded data;
transmit the generated RF signal to an external clinician computing device through the RF transmit antenna.

12. The intervertebral implant of claim 11, wherein the body includes a through hole in communication with the fusion aperture and the cartridge is at least partially disposed in the through hole.

13. The intervertebral implant of claim 11, wherein the fusion aperture is an elongate aperture defining a central longitudinal axis, and the cartridge extends along the central longitudinal axis.

14. The intervertebral implant of claim 13, wherein the cartridge extends through the fusion aperture.

15. The intervertebral implant of claim 13, wherein the cartridge extends into the fusion aperture in a direction lateral to the central longitudinal axis.

16. The intervertebral implant of claim 11, wherein the cartridge includes a biofilm sensor for detecting the presence of biofilm, the biofilm sensor including a signal transmitter and signal receiver overlying an external surface of the cartridge.

17. The intervertebral implant of claim 16, wherein the cartridge includes circuitry for analyzing attenuation of a signal transmitted by the signal transmitter and received by the signal receiver.

18. The intervertebral implant of claim 17, wherein the circuitry is configured to generate a range of RF signals sweeping through a defined frequency range or amplitude range, and detect the presence of biofilm based on identifying at least a threshold change in amplitude or frequency of the received signal at one or more defined marker frequencies or defined marker amplitudes.

19. The intervertebral implant of claim 11, wherein the at least one cartridge includes a plurality of cartridges with each disposed in a respective body and that communicate with each other in a mesh network.

20. The intervertebral implant of claim 19, further comprising a hub cartridge configured to be implanted in the patient and operative to communicate with all of the cartridges to collect recorded data from the cartridges for transmission to the clinician computing device.
